(19) United States
(12) Patent Application Publication
(10) Pub. No.: US 2004/0175340 A1
(43) Pub. Date: Sep. 9, 2004

(54) Cold wax hair removal (depilatory) compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/248,925

(22) Filed: Mar. 3, 2003

(51) Int. Cl. A61K 7/06; A61K 7/15

(52) U.S. Cl. 424/70.1

(57) ABSTRACT

I have discovered cosmetic cold-wax hair removal (depilatory) compositions suitable for site-specific face or body applications with the following attributes: (1) A pre-heating or microwave step is not necessary, (2) Complete hair removal is achieved in a single application on a specific site or area, (3), No (or minimal) skin irritation is experienced, (4) Bond to hair preferentially, and not to skin, (5) Provide lubricity and emolliency to skin, (6) Can be applied either directly on the skin or, preferably, on a piece of precut fabric, plastic or paper, which is then applied to specific site where hair removal is desired. Alternatively, dispensing applicators can also be used that can be filled for delivery at the specific site where depilation is desired. (7) Require minimal clean-up with water or common make-up removal compositions, and (9) Additional skin and hair beneficial ingredients, such as hair growth retardants, anti-irritants, topical pain relief agents, antioxidants, skin soothing agents, skin cooling agents, emollients, moisturizers, topical anesthetics, colorants, botanical extracts, fragrances, and such can be included.

### BACKGROUND OF INVENTION

Various compositions and methods for removing hair (depilation) are known. For example, hair can be removed by electrolysis, by chemical action, or by physical removal. Physical removal generally involves the use of wax or tacky tape. Generally, two types of wax for depilatory purposes are used for hair removal, a hot wax or a cold wax. Cold wax is gummy at room temperature, whereas hot wax is hard to medium hard at room temperature. The hot wax compositions are typically heated above their melting point and coated onto the skin. Upon solidification, hair becomes trapped in the wax and is removed when the wax is peeled off the skin. Cold wax compositions do not require such a pre-heating or microwave step prior to their application for depilation.

Various depilatory compositions which utilize physical removal action are described in, for example, U.S. Pat. Nos. 6,478,493; 5,847,363; 5,698,187; 5,848,850; 5,846,326; 5,158,765; 5,154,919; 5,840,765; 5,470,563; 4,832,949; 2,091,313 and U.S. Patent Application 20030002912, among others.

One recognized problem with the use of hot waxes and rosin-based compositions is that they require a pre-heating or microwave step prior to their application, and they also inherently incorporate components or impurities that cause irritation that may result in redness, swelling or blisters. Such problems have been discussed in U.S. Patent Application 20010001660 (Romero et al.), and several of the above referenced patents seek to address this problem by utilizing elastomers, or organo-polysiloxane, or by avoiding these ingredients entirely. Romero et al. disclose a hair removal composition that includes bees wax, rosin and an inhibiting salt for inhibiting the irritating agents present primarily as impurities in bees wax and rosin. The composition is heated, applied to the skin and allowed to solidify, embedding hair follicles therein. After solidification, the composition is peeled off the skin.

As can be appreciated by those who are versed in this art that such depilatory compositions that require heating or microwave before their direct application on the skin are not convenient. Moreover, they can cause injury or burns, since the heating temperatures can not be adequately controlled or guessed prior to the application of the heated product on the skin. Furthermore, such compositions are highly sticky that adhere strongly to both hair and skin. During the hair removal step usually the product is applied directly to skin and a fabric or paper is placed over it. After the product has cooled and solidified on the skin, the fabric or paper is pulled away from the skin in a fast motion, thus removing hair. During this process, some epidermal layers of skin are also removed or stripped away, exposing lower layers of skin and initiating inflammatory reaction from prostaglandin”s formation. Actually, this is the principal reason for the development of skin irritation, and not minor impurities in the composition. This fact has not been recognized by prior art. For example, prior art has focused mainly on the use of anti-irritants to circumvent this problem of skin irritation, as disclosed in U.S. Pat. No. 5,470,563 (Tanaka et al.).

In order to solve the problem of damage to skin from such heated depilatory compositions, several “cold-wax” hair removal compositions have been disclosed. Most such compositions are based on sugar or sugar derivatives.

U.S. Pat. No. 5,158,765 (Qasem) discloses a depilatory composition for the removal of hair that is composed of a mixture of sugar, water and aspirin. This mixture is heated to dissolve the solute materials, and then allowed to cool so as to form a soft, pliable composition which can be manually applied to the skin. The composition firmly adheres to the hair with which it comes in contact, and by quickly drawing the applied material away from the skin, will cause the hair to be removed from its roots. One problem from such compositions that are directly applied to skin is that usually an area larger than the area where depilation is needed is coated with such compositions, requiring a more extensive cleanup step after hair removal. Another problem is that such cold-wax compositions based on sugar or sugar derivatives do not remove all the hair in a single step, requiring two or more applications in the same area. This causes waste of product, additional skin irritation, and inconvenience.

Among other similar sugar-based compositions, U.S. Pat. No. 5,698,187 (Naggiar) discloses depilatory composition for the removal of hair from the human body that is composed of a mixture of maltodextrin, sucrose, water and citric acid. This mixture is heated to dissolve the solute materials, and then cooled to form a soft, pliant composition which can be manually applied to the skin.

U.S. Pat. No. 4,842,610 (Gordon et al.) discloses hair removal compositions that comprise 90 to 99.5% corn syrup and 0.5% to 10% added water by weight. The compositions are used by applying them to the surface of the skin in a hairy area, pressing a sheet of paper or other fibrous material against the area and subsequently lifting the sheet of fibrous material or peeling it off the skin surface.

U.S. Pat. No. 4,832,949 (Royal) discloses a depilatory composition for removal of hair from the human skin that is made up of a mixture of honey, sugar and citric acid which mixture is heated to a predetermined temperature level, then allowed to cool so as to form a highly viscous, wax-like composition which can be applied manually in slender strips to the skin.

It is commonly recognized that sugar-based compositions do not remove hair adequately in a single application, as mentioned above. The most preferred depilatory compositions are based on rosin or rosin derivatives, or polymers. However, such compositions must be applied in hot state, causing the problems of inconvenience, burns, skin irritation, extensive cleanup after use, and such as mentioned earlier. Sugar and sugar derivatives based compositions, on the other hand, contain substantial amounts of water, or alcohol, or mixtures of water and alcohol, which tend to evaporate each time a bottle is opened for product application, thus resulting in the crystallization of sugar or sugar derivatives from such compositions. Such crystallizations cause a loss of the hair removing power of such compositions, and also make it harder for the bottle to be opened for product use. U.S. Pat. No. 6,417,346 (Salome et al.) further discusses such sugar solution crystallization problems. Additionally, a “dry-down” period is required for such sugar and sugar derivatives based compositions after product applications to let water or alcohol partially evaporate, and before the depilation step is completed. This “dry-down” period can be from 5 minutes to 20 minutes, or even longer. The product does not gain sufficient “stickiness” to remove hair if this “dry-down” period is not observed.

It would thus be advantageous if a depilatory composition can be made that has the following properties: (1) It is applied cold without requiring any pre-heating step, and (2) It is made from rosin or rosin derivatives or polymers (i.e. not sugar or sugar derivatives based because such compositions do not perform adequately, as mentioned before), and (3) It can be applied either directly on the skin or, preferably, it is applied on a piece of fabric, plastic, or paper first, which is then applied to skin for hair removal. Alternatively, a dispensing applicator can also be used that can be filled for delivery at the specific site where depilation is desired. Such methods allow the application of product only in the area desired for hair removal. Although pre-cut strips of plastic or fabric pre-coated with depilatory compositions are already available to consumer, such products are expensive and can not be cut into desired shaped since they are already coated with a highly sticky composition. If consumers can pre-cut a piece of fabric, plastic or paper to a size and shape desired for depilation, then coat such a piece with a depilatory composition that does not require any pre-heating and does not cause skin irritation, then such a depilatory composition should be of high consumer demand due to its convenience, cost, ease of application, safety, and comfort.

Also, as mentioned earlier, the skin irritation is caused mostly by the stripping of the upper layers of skin from such sticky depilatory compositions. It would be ideal if such compositions can adhere preferentially to hair, and not skin. Although not bound by any theory, it should be possible to achieve this in such a selective manner. It is well known that hair contains a much larger amount of sulfur-containing amino acids, cysteine and cystine that are chemically reactive in forming new bonds. Skin, on the other hand, is known to contain a large amount of acidic amino acids that gives skin surface an acidic pH, hence an anionic character. If a depilatory composition can be devised to bond more strongly with such sulfur groups of hair and cause less strong bonding with skin surface taking advantage of skin”s acidic surface, then such a bonding would provide more selective adherence to hair surface, thus causing hair removal much easier, and causing much less disruption of the upper layers of skin. Alternatively, if a composition can be devised that places an emollient or protective film on the skin but still binds with hair sufficiently so hair is removed and skin is not affected due to the easier sliding-off of the depilatory composition during hair removal step. Such features would provide less damage to the upper layers of skin and hence much reduced or no skin irritation. It is the purpose of the present invention to provide such depilatory compositions.

### SUMMARY OF INVENTION

The present invention discloses cosmetic cold-wax hair removal (depilatory) compositions suitable for site-specific face or body applications that provide the following benefits: (1) A pre-heating or microwave step is not necessary, (2) Complete hair removal is achieved in a single application on a specific site or area, (3), No (or minimal) skin irritation is experienced, (4) Bond to hair preferentially, and not to skin, (5) Provide lubricity and emolliency to skin, (6) Can be applied either directly on the skin or, preferably, on a piece of precut fabric, plastic or paper, which is then applied to specific site where hair removal is desired,. Alternatively, dispensing applicators can also be used that can be filled for delivery at the specific site where depilation is desired. (7) Require minimal clean-up with water or common make-up removal compositions, and (9) Additional skin and hair beneficial ingredients, such as hair growth retardants, anti-irritants, topical pain relief agents, antioxidants, skin soothing agents, skin cooling agents, emollients, moisturizers, topical anesthetics, colorants, botanical extracts, fragrances, and such can be included.

Based on the above properties required for a highly desirable cold-wax depilatory composition, the present invention discloses such compositions that are based on: (1) A hair binding agent or composition, and (2) A skin slip or skin protectant agent or composition, and (3) A liquid carrier composition, and, optionally (4) A hair growth retardant, skin soothing, anti-irritant, topical analgesic, antioxidant, UV absorber, or other such skin or hair beneficial composition, or combinations thereof.

### DETAILED DESCRIPTION

A cold-wax depilatory composition that provides the following benefits would be highly desirable for consumer markets: (1) A pre-heating or microwave step is not necessary, and (2) Complete hair removal is achieved in a single application on a specific site or area, and (3), No (or minimal) skin irritation is experienced, and (4) Compositions bind to hair preferentially, and not to skin (thus further reducing or eliminating skin irritation), and (5) Compositions provide lubricity and emolliency to skin, and (6) Composition can be applied either directly on the skin or, preferably, on a piece of precut fabric, plastic or paper, which is then applied to hair to be removed, and (7) Requires minimal clean-up with water or common make-up removal compositions, and (8) Additional skin beneficial ingredients, such as anti-irritants, skin soothing agents, skin cooling agents, emollients, moisturizers, antioxidants, anti-wrinkle additives, anti-aging compositions, skin smoothing agents, UV absorbers, topical anesthetics, colorants, botanical extracts, fragrances, and such can be included.

The present invention discloses cold-wax depilatory compositions that accomplish the above objectives. These compositions are based on: (1) A hair binding agent or composition, and (2) A protective skin coating agent or composition, and (3) A liquid carrier composition, and, optionally (4) A hair growth retardant, skin soothing, anti-irritant, topical analgesic, antioxidant, UV absorber, or other such skin or hair beneficial composition, or combinations thereof.

It is well known that hair contains a much larger amount of sulfur-containing amino acids, cysteine and cystine that are chemically reactive in forming new bonds. Skin, on the other hand, is known to contain a large amount of acidic amino acids that gives skin surface an acidic pH, hence an anionic character. Such differential physical and chemical properties of hair and skin have not been utilized in the prior art depilatory compositions for selective removal of hair without damage to skin by undesirable excessive exfoliation from such compositions that can cause skin irritation and infection.

Surprisingly and unexpectedly, it has now been discovered that hair can be bound selectively, while skin is provided with a lubricating film for slip and protective emolliency that does not reduce the binding of hair with depilatory compositions of the present invention. The selection of hair bonding agent or composition is based on the fact that hair contains a substantially higher amount of certain sulfur-containing amino acids, such as cysteine and cystine, in its protein structure. Such sulfur amino acids are reactive that can be bound with certain resinous or polymeric compositions preferentially. This would facilitate easier removal of hair without causing any significant damage to the upper layers of skin that has been responsible for causing skin irritation in so many prior art depilatory (hair removal) compositions that require a heating or microwave step. Although several sugar or sugar derivatives based “cold-wax” compositions are known that do not require a pre-heating or microwave step, such compositions (although they do offer an additional convenience of easy cleanup with water) usually require more than one application on the same site where depilation is desired. It is commonly known that certain rosin, rosinates, and polymers do provide the convenience of complete hair removal in a single application, such compositions usually require pre heating or microwave step. When such compositions are made thinner in order to avoid a pre-heating step, such “diluted” compositions do not provide adequate hair removal in a single treatment, just like the problem with sugar based “cold-wax” depilatory compositions.

Surprisingly, I have discovered that many rosin, rosinates, and polymeric hair binding compositions can now be incorporated in a mobile liquid composition that does not require a pre-heating or microwave step, yet such compositions still remove all hair in a single treatment. Moreover, such compositions can also be applied first to a fabric, paper, or plastic piece pre-cut to a desired shape and size, which is then applied to hair for depilation. This offers more convenience and precise treatment of the depilation area. Moreover, the soaking of the depilatory compositions of the present invention first on such fabric, plastic, or paper surfaces does not diminish their hair removal power.

A wide selection of rosin, rosinates, and polymers can be utilized either alone or in combination. The examples include, but not limited to Wood Rosin, Glyceryl Rosinate, Glyceryl Hydrogenated Rosinate, Polyethylene Glycol Rosinate, Polyethylene Glycol Hydrogenated Rosinate, Pentaerythritol Rosinate, Pentaerythritol Hydrogenated Rosinate, Polyvinyl Alcohol, Polyvinyl Acetate, Polyvinyl Esters, PVP, PVP/PVA Copolymers, Dimerized Rosin, Esters of Dimerized Rosin, Modified Wood Rosin, Esters of Modified Wood Rosin, Polyol Ester Rosinate, Polyterpene Resins, Esters of Hydrogenated Modified Rosin; (Hydrogenated Rosins covers both fully and partially hydrogenated forms), Polyethylene, Polypropylene, Polystyrene, Polyisobutylenes, EVA Resins, Block Copolymers, Polyvinyl Ethers, Polyacrylics, Polyvinyl butyral, Polyamides, Aromatic Hydrocarbon Resin, Cellulose Acetate, Ethyl Cellulose, Cellulose Acetate Butyrate, Ethyl Hydroxyethylcellulose, Nitrocellulose, Alkyl Resins, Rosin Ester Resins, Hydrocarbon waxes, Natural Waxes, Shellac, Natural Rubber, Styrene-Butadiene Rubber, Nitrile Rubber, Butyl Rubber, Polychloroprene Rubber, Chlorinated Rubber,The compositions of the present invention also include at least one protective skin coating composition. The purpose of such compositions is to provide slip during the pulling of the fabric, plastic, or paper for hair removal, thus protecting any damage to the upper layers of skin during depilation process. This makes the removal of the composition from the skin surface easier, thus eliminating or significantly reducing any damage to the outer layers of skin that can cause the skin irritation or infection, as commonly experienced with other rosin, Rosinate, or polymer based depilatory compositions.

The protective skin coating agent can be selected from, but not limited to, natural oils, natural butters, natural waxes, synthetic oils, synthetic waxes, silicone oils, silicone waxes, silicone elastomers, Silicone Rosinates, Alkoxylated Silicone Rosinates, organic siloxanes, and their cross polymer (e.g., dimethicone, dimethicone copolyol, cetyl dimethicone copolymer, cetyl dimethicone, stearyl dimethicone, stearoxydimethicone, behenoxydimethicone, alkyl methicone, amodimethicone, dimethicone alkyl betaine, cyclomethicone, polydimethylsiloxane, diphenyidimethyl polysiloxane, silicone elastomers, cyclomethicone and dimethicone crosspolymer, Jeesilc 6056, Dow Corning 2501, modified celluloses, hydroxyethyl cellulose, ethyl cellulose, carboxymethyl cellulose, ethyl hydroxyethyl cellulose, xanthan gum, gellan gum, guar gum, cationic polymers, Aristoflex AVC (Ammonium Acryloyldimethyltaurate/VP Copolymer), Structure Plus and Structure XL (Acrylates/Aminoacrylates/c10-30 Alkyl PEG-20 Itaconate Copolymer ), Carbomer, Carbopol ETD 2020 (Acrylate C10-30 Alkyl Acrylate Crosspolymer), Rheocin (trihydroxystearin), Hydramol PGDS (PEG 90 Diisostearate), C24-28 Alkyl Dimethicone, Behenyl alcohol, quaternary ammonium compounds, sugar, sugar derivatives, corn syrup, honey, maltodextrin, and combinations thereof.

The third component of the present invention is the inclusion of at least one liquid binder composition that binds with all other ingredients of the depilatory composition. The most important feature is that such liquid binder composition provides the depilatory composition as a mobile, easy to pour and spread liquid form that does not require any pre-heating or microwave step prior to such application. Moreover, such liquid binder does not absorb into fabric, paper, or plastic piece on which the depilatory composition is in contact during the depilation process. Moreover, the liquid binder composition does not evaporate, thus causing the crystallization of its components that can cause the loss of hair removal power of such compositions. It is to be noted at this point that all of sugar and sugar derivatives based compositions contain substantial amounts of water, alcohol, or water and alcohol mixtures that easily evaporate from their containers thus causing the crystallization of such sugar and sugar derivatives resulting in the total of the hair removal capability of such sugar and sugar derivatives based compositions.

The preferred liquid binder agent or composition can be selected from, but not limited to, natural oils (such as almond oil, castor oil, linseed oil, sunflower seed oil, mango oil, coconut oil, corn oil, vegetable oil, sesame seed oil, jojoba oil, pistachio nut oil, macadamia nut oil, and such), natural butters (such as mango butter, coco butter, almond butter, shea butter, aloe butter, sal butter, kokum butter, butters made from hydrogenated natural oils, and such), polyethylene glycol, polypropylene glycol, polybutylene glycol, methylpropanediol, various carbowax derivatives, propylene glycol, butylene glycol, hexylene glycol, ethoxydiglycol pyrrolidone, N-Methyl pyrrolidone, DMSO, and combinations thereof.

A number of hair growth retardants are also known. It is commonly known that such hair growth retardants usually provide better action if applied to skin immediately after depilation process. The inclusion of such hair growth retardants in the depilatory compositions should provide additional advantages in that the depilatory step would be required less frequently. The inclusion of such hair growth retardants is not possible in the compositions that require a pre-heating step, as most hair growth retardants are not stable under the conditions of heating. Since the compositions of the present invention do not require a pre-heating or microwave step, such hair growth retardants can be included. Examples of such hair growth retardant compositions that can be included are, but not limited to, U.S. Pat. No. 5,908,867 (Henry et al.), U.S. Patent Applications 20030036561 (Stykzynski et al.), 20030026776 (Shibuta et al.), 20020197290 (Di Pierro), 20020146404 (Tsuji et al.), U.S. Pat. Nos. 6,414,017; 6,239,170; 6,248,751 (Ahluwalia et al.), U.S. Pat. No. 6,407,056 (Sieberg et al.), U.S. Pat. No. 6,375,948 (Tsuji et al.), U.S. Pat. Nos. 6,299,865; 6,235,737 (Styczynski et al.), and U.S. Pat. Nos. 6,218,435; 6,121,269 (Henry et al.) among others, and various commercially marketed compositions, such as Pilinhibit, Capislow (Sederma/Croda).

Additional skin and hair beneficial ingredients or compositions can also be included in the compositions of the present invention. Since a pre-heating or microwave step is not necessary, such additives remain stable and chemically unaltered. The examples include, but not limited to, skin soothing agents, antioxidants, topical anesthetics, antibacterial agents, emollients, moisturizers, skin surface cleansing agents, botanical extracts, perfumes, colorants, preservatives, color stabilizers, antioxidants for rancidity control and such, and combinations thereof can also be included in amounts that are safe and sufficient for their intended benefits and functions.

Although the compositions of the present invention do not cause irritation or pain during depilation process, it may be desirable to include skin cooling and skin numbing agents for some consumers who have delicate, sensitive skin. The examples of such ingredients that can be selected for this purpose includes, but not limited to, menthol, menthol esters, methyl salicylate, camphor, benzocaine, dibucaine, dyclonine, lidocaine, pramoxine, tetracaine, ephedrine, epinephrine, phenylephrine, and their derivatives, and combinations thereof.

### EXAMPLES

The following examples are presented to illustrate presently preferred practice thereof. As illustrations they are not intended to limit the scope of the invention. All quantities are in weight %.

### Example 1

Ingredient % (1) Glyceryl Hydrogenated Rosinate 65.0 (2) Castor Oil 19.8 (3) Siliconyl Beeswax 15.0 (4) Tetradibutyl pentaerythrityl Hydroxyhydrocinnamate (a color stabilizer) 0.2 Procedure. Mix all components and heat at 90 to 95° C. to a clear liquid. Cool to room temperature. A clear, light amber mobile liquid is obtained. It is applied to skin or pre-cut fabric, plastic, or paper without any pre-heating or microwave.

### Example 2

Ingredient% (1) Glyceryl Hydrogenated Rosinate 70.0 (2) Polypropylene Glycol 29.8 (3) Tetradibutyl pentaerythrityl Hydroxyhydrocinnamate 0.2 Procedure. Mix all components and heat at 90 to 95° C. to a clear liquid. Cool to room temperature. A clear, light amber mobile liquid is obtained. It is applied to skin or pre-cut fabric, plastic, or paper without any pre-heating or microwave.

### Example 3

Ingredient % (1) Glyceryl Hydrogenated Rosinate 65.0 (2) Castor oil 30.8 (3) Mango butter (and) shea butter (and) sal butter (and) grape seed oil (and) kiwi seed oil 4.0 (4) Tetradibutyl pentaerythrityl Hydroxyhydrocinnamate 0.2 Procedure. Mix all components and heat at 90 to 95° C. to a clear liquid. Cool to room temperature. A clear, light amber mobile liquid is obtained. It is applied to skin or pre-cut fabric, plastic, or paper without any pre-heating or microwave.

### Example 4

Ingredient% (1) Glyceryl Hydrogenated Rosinate 70.0 (2) Castor Oil 27.8 (3) Polydimethylsiloxane 2.0 (4) Tetradibutyl pentaerythrityl Hydroxyhydrocinnamate 0.2 (5) Hydroxystearic acid 1.0 Procedure. Mix all components and heat at 90 to 95° C. to a clear liquid. Cool to room temperature. A clear, light amber mobile liquid is obtained. It is applied to skin or pre-cut fabric, plastic, or paper without any pre-heating or microwave.

### Example 5

Consumer Testing of Compositions of Example 1 to Example 4. A six person panel, all female, tested the compositions of example 1 to example 4 by both direct application on skin and also the application of the product first on fabric pieces, then the application of such coated fabric pieces on skin for depilation. The ratings, based on ease of product application, completeness of hair removal in a single treatment, ease of cleanup after use, and amount of skin irritation, from most preferred to least preferred, follow: Example 3=Example 4>Example 2>>Example 1.

Two commercially purchased products, a sugar based “cold-wax” product, and rosin based “microwave” product, were also used as controls. All Examples, from 1 to 4, were preferred over the above two commercially marketed products used as “controls” tested by the same group of panelists. These tests were conducted all at the same time.

### Example 6

Ingredient % (1) Polyvinyl alcohol (Celvol 502) 45.0 (2) Ethoxydiglycol 20.0 (3) Water 31.5 (4) Ethyl cellulose 1.0 (5) Preservatives 0.5 (6) Menthol 2.0 Procedure. Mix (1) to (4) and heat at 90 to 95° C. till a clear solution is obtained. Cool to 40 to 45° C., and add (5) and (6) and mix. Cool to room temperature. A clear mobile liquid composition is obtained. It is applied to skin or pre-cut fabric, plastic, or paper without any pre-heating or microwave. Any residues left on skin after hair removal can be cleaned with warm water. Menthol provides a cooling, soothing sensation after hair removal. Also, although this composition contains a significant amount of water, it does not dry or crystallize upon storage due to water-retentive properties of the composition.

### Example 7

Ingredient % (1) Polyvinyl alcohol (Celvol 502) 45.0 (2) Ethoxydiglycol 20.0 (3) Water 30.0 (4) Ethyl cellulose 1.0 (5) Preservatives 0.5 (6) Benzocaine 2.0 (7) Pilinhibit 1.5 Procedure. Mix (1) to (4) and heat at 90 to 95° C. till a clear solution is obtained. Cool to 40 to 45° C., and add (5) to (7) and mix. Cool to room temperature. A clear mobile liquid composition is obtained. It is applied to skin or pre-cut fabric, plastic, or paper without any pre-heating or microwave. Any residues left on skin after hair removal can be cleaned with warm water. Benzocaine provides a numbing, soothing sensation after hair removal. Pilinhibit provides hair growth retardant benefits. Also, although this composition contains a significant amount of water, it does not dry or crystallize upon storage due to water-retentive properties of the composition.

I claim:

1. A cosmetic cold-wax depilatory composition suitable for facial or body application comprising:
(i) At least one hair binding composition, and
(ii) At least one protective skin coating composition, and
(iii) At least one liquid binder composition that binds with the ingredients of the compositions in (i) and (ii).

2. A composition according to claim 1 wherein hair growth retardants are also included in safe and efficacious amounts.

3. A composition according to claim 1 wherein antioxidants are also included in safe and efficacious amounts.

4. A composition according to claim 1 wherein topical pain relief compositions are also included in safe and efficacious amounts.

5. A composition according to claim 1 wherein additional skin and hair beneficial compositions, such as emollients, moisturizers, skin surface cleansing agents, botanical extracts, skin soothing agents, antibacterial agents, anti-wrinkle additives, anti-aging compositions, skin smoothing agents, UV absorbers, perfumes, colorants, preservatives, color stabilizers, mica pigments, antioxidants for rancidity control, and such are also included in the depilatory composition.

6. A composition according to claim 1 wherein the hair binding composition is from 10% to 75% of the depilatory composition.

7. A composition according to claim 1 wherein protective skin coating composition is from 0.1% to 10% of the depilatory composition.

8. A composition according to claim 1 wherein the liquid binder composition is from 10% to 90% of the depilatory composition.

9. A composition according to claim 1 wherein such depilatory compositions can be filled in dispensing applicators of various sizes and delivery modes.

10. A composition according to claim 1 wherein such depilatory compositions can be pre-coated on various dispensing surfaces such as fabric, non-woven fabric, paper, plastic, and such in various sizes and shapes.
